(19) United States
(12) Patent Application Publication
(10) Pub. No.: US 2015/0368681 A1
(43) Pub. Date: Dec. 24, 2015

(54) Dehydroascorbic Acid Process

(71) Applicant: Douglas Quinten Kitt, SLC, UT (US)

(72) Inventor: Douglas Quinten Kitt, SLC, UT (US)

(21) Appl. No.: 14/728,889

(22) Filed: Jun. 2, 2015

Related U.S. Application Data
(60) Provisional application No. 62/015,808, filed on Jun. 23, 2014.

(51) Int. Cl. C12P 17/04; C07D 307/62; A23L 1/302

(52) U.S. Cl. CPC C12P 17/04; A23L 1/302; C07D 307/62; A23V 2002/00

(57) ABSTRACT

Processes to produce dehydroascorbic acid at the time of use are provided, so that this unstable form of vitamin C can be conveniently utilized for dietary purposes. Products produced by these processes are also described.

### BACKGROUND

1. Field of Invention

This invention relates to the field of improved absorption of orally ingested vitamin C, and more specifically to the form of vitamin C known as dehydroascorbic acid (DHAA). DHAA is the naturally-occurring oxidized form of vitamin C, and has many unique properties as compared to the reduced form, ascorbic acid (AA). Many of these properties are described in U.S. Pat. No. 8,324,269 which is incorporated herein in its entirety by reference.

Absorption of orally ingested vitamin C into the cells of the gut is mediated by transport proteins in the cell membrane. AA or ascorbate ion absorption occurs via various members of the family of transport proteins known as SVCT, whereas DHAA is absorbed utilizing members of the GLUT family. GLUT transporters are a type known as passive transporters, which in general means that they transport more rapidly than the type known as active transporters. Also passive transport is not a saturable mechanism as is active transport. Furthermore, GLUT transporters are abundant in the nutrient-absorbing cells lining the gut because these transporters also transport common sugars like glucose. Taken together, these facts mean that DHAA can be absorbed much more rapidly and efficiently when orally ingested than can the more common AA, because the SVCT transport proteins are active transporters that can be saturated and are less abundant in the gut. Therefore oral consumption of DHAA has great advantages as compared to the oral consumption of AA that is found in common vitamin C supplements, including greater bioavailability of larger doses, more rapid absorption, and higher vitamin C blood levels post consumption.

2. Prior Art

Background art includes U.S. patent application Ser. No. 10/572,790 (Mar. 21, 2006) of Gassier (now abandoned). Gassier describes a method of producing DHAA by supplying multi-part components that can be combined to cause the oxidation of ascorbate to DHAA for a cosmetic purpose. But Cassier's invention requires at least one cosmetic ingredient, and is silent as to the use of the product for oral ingestion.

### SUMMARY OF INVENTION

DHAA is a notoriously unstable chemical compound; in aqueous solution, particularly at neutral or basic pH and at warmer temperatures, it undergoes rapid hydrolysis to 2,3-diketogulonic acid (DKG) and irreversibly loses its vitamin C activity. Thus it is difficult and expensive to provide to consumers for convenient oral consumption. I have now found that DHAA can be provided conveniently and economically by creating it at the time of use, such that extended storage is not necessary. I have found that DHAA can be rapidly created by a simple method using AA (or some other form of reduced ascorbate, such as sodium ascorbate) as the substrate, using oxygen as a second substrate, and using an enzyme called Ascorbic Acid Oxidase that is found naturally in many plants, and is particularly abundant in zucchini fruits. Oxygen may be provided by stirring the mixture in air, or by bubbling air through the mixture. Methods and the products produced by these methods are described here.

### DESCRIPTION OF EMBODIMENTS

In a preferred embodiment, certain fresh, raw vegetables that contain the enzyme Ascorbic Acid Oxidase can be used to rapidly produce DHAA by oxidation of AA in a puree of the vegetable. For example, zucchini squash is known to contain extremely high levels of AAO. See Example 1.

In another embodiment, the peelings from the skin and fleshy middle layer (the epicarp and mesocarp) of zucchini fruit were used. It is known that the AAO enzyme is found at higher concentrations in the epicarp and outer mesocarp than in the endocarp and seeds of the zucchini fruit and other fruits and vegetables. By using the outer portions, a higher concentration of AAO can be obtained in a puree. See Example 2.

In another embodiment, AA was added in increments, or stages, to zucchini puree. It is known that AAO has an optimal pH range for activity, generally between about pH 4 and pH 9. It is also known that extreme pH values in solutions can prevent enzyme reactions from proceeding and even destroy the enzyme activity altogether. One way to take advantage of the powerful AAO enzyme activity of zucchini without killing the reaction with too much AA acidity is to add the AA in increments instead of all at once. As the AA is oxidized, the pH of the puree goes up since DHAA is not acidic. Then additional AA can be added. This process can be repeated many times, keeping the pH of the puree within the optimal pH range while accumulating very high concentrations of DHAA. See Example 3.

Since DHAA is much less stable at neutral pH than at acidic pH, it is desirable to maintain the pH of a puree below about 7.0 during the oxidation process to avoid hydrolysis of the DHAA after it is produced. It is surprisingly found that a puree of zucchini is more acidic than pH 7.0 naturally, and it is also surprisingly discovered that as more AA is added, the pH of the final puree is more acidic. See Example 3.

In addition to AA, other forms of reduced vitamin C such as ascorbate ion can also be oxidized by AAO, including such salt forms as sodium ascorbate and calcium ascorbate, and chemical derivatives of AA such as ascorbyl phosphate and ascorbyl palmitate. All forms of reduced AA, including ascorbate and oxidizable derivates of AA may be employed in this process. I have found that concentrations of reduced vitamin C from as low as 0.1% w/w to as high as 20% w/w may be effectively oxidized according to the embodiments described here.

Acids, bases and buffers can be added to adjust the pH to desirable levels, before during or after the oxidation process.

Recovery of DHAA in the product is about 95%. See Example 3.

Product can be stored for at least 13 days frozen with minimal loss of DHAA. Therefore the product can be distributed in the frozen state. See Example 3.

The oxidized product can be further combined with polyol such as glycerin for longer stability, or flavoring, or texture adjustment.

Zucchini fruit, other vegetable matter containing AAO, or parts thereof can be frozen to preserve the natural AAO activity.

Dried or freeze-dried zucchini fruit or parts thereof can be used because enzyme activity is preserved on drying. AAO, and other enzymes, may be extracted from vegetables to create partially purified or highly purified extracts. These extracts may be dried or crystallized, or stabilized by other means to protect the enzyme activity. The dried vegetable matter or purified extracts can be combined with water and reduced vitamin C in a solution to create DHAA.

Agents that solubilize enzymes that are localized in the cell walls or cell membranes of plants can enhance the rate or reliability of the enzyme reaction or enhance the recovery of enzymes in extracts. Such agents include surfactants and detergents, chaotropes, and lytic enzymes. Representative agents include non-ionic detergents such as Triton-X, SDS, and lytic enzymes that specifically degrade the cell wall or cell membrane, including various proteases, pectinases, cellulases and hemicellulases.

Other agents can enhance enzyme activity or recovery and include agents to control ionic strength, osmotic strength, and the activity of nucleases and proteases.

Peroxidases and catalases in vegetables help stabilize AAO to prevent inactivation and exhaustion of the AAO during reaction. Using certain vegetables therefore provides the unexpected advantage of providing both AAO and peroxidase.

Numerous other vegetables contain AAO activity and/or catalase and/or peroxidase activity including Arabidopsis, Brassica, Cucumis, Cucurbita, Myrothecium, Nicotiana, Oryza, Sinapis, Titicum species, cabbage, squashes, pumpkins, peas, string beans, Lima beans, sweet corn, Swiss chard, carrots, parsnips, and spinach. Other vegetables can be used in this process to produce DHAA product. Combinations of different vegetables can be used to optimize the AAO/catalase/peroxidase ratios and to optimize the pH of a puree, or a solution, or a suspension of vegetable matter. Synthetic AAO enzyme may also be produced by methods known in the art, and may also be used instead of natural vegetable matter containing AAO.

A desirable product can be made by combining fruit(s) or vegetable(s) that is/are naturally high in AA content with fruit(s) or vegetable(s) that is/are naturally high in AAO content, to create a completely natural product containing a high content of DHAA.

A product can be made that is dried vegetable or vegetable parts plus AA or other form of reduced ascorbate, either mixed together or separately packaged, for rehydration and mixing with air to produce DHAA. This has the advantage of a stable enzyme, providing catalase and/or peroxidase, allowing proportions of enzyme vs. AA and time of reaction to be pre-determined, and assuring the oxidation reaction will work in the hands of the consumer.

A product can be in the form of a kit, including such additional components as a redox indicator, mixing vessel, instructions, gelatin, pH adjusters, mixing tools, etc.

The dried vegetable and AA, or the mixture of the two, can be distributed in pouches, unitized doses, tablets, capsules, or other convenient containers or easy to handle forms, including pre-measured amounts.

Gelatin capsules would provide gelatin for the reaction, which stabilizes AAO.

Copper can be added to a puree or solution containing AAO to enhance the activity and stability of the enzyme. Copper can be provided in the form of a soluble copper salt, or a solution containing dissolved copper, or by mixing the solution in a container made of copper. I have found that copper in the concentration of about 0.01 mg/dl up to about 20 mg/dl is effective for increasing the activity of the enzyme, and extending the period of time that the enzyme remains active before it is exhausted. Although copper ions alone are known in the art to increase the oxidation rate of AA in solution, this rate is much slower than the rate at which oxidation occurs in the presence of AAO. It is an unexpected discovery that the AAO enzyme activity is greatly increased, i.e. to a much higher rate than would be expected due to the copper activity alone, by the presence of copper ions in the concentrations described. It is an unexpected and surprising discovery that the enzyme remains active for longer periods of time when copper ions are added. This effect may be attributable to providing additional copper ions that are available to restore the copper ions found naturally in the AAO enzyme, as it has been reported that copper ions are exchanged between the enzyme and the solution during the oxidation process. The applicant does not wish to be held to this explanation, however, as other explanations are possible.

AA concentration in solution is commonly measured as the reducing activity of the solution using starch-iodine titration methods that are well-known in the art. Modification of the starch-iodine titration method can be used to detect AA in a vegetable/AA puree and therefore provide a process and a product for determining if and when AA has been converted to DHAA. A product or reagent for this process can be described as a redox indicator reagent.

A redox reagent can be made by combining iodine, iodide, and starch in solution. Such redox reagent can be optimized in concentration, or utilized in various amounts, to indicate various concentrations of AA.

Starch-iodine redox reagent can be dried on paper or immobilized by other methods to provide a convenient test such as a paper test strip or pad.

A redox reagent utilizing a different chemical or chemicals known to be indicators of reduction-oxidation potential can be used.

Progress of oxidation of AA to DHAA may be monitored by pH measurements.

A business method can involve the production of “vegetable smoothies” containing DHAA in retail outlets, or licensing or franchise.

A business method can involve the licensing of the rights or directions to produce DHAA.

An apparatus optimized to puree the vegetable, or enhance air or oxygen incorporation, or otherwise improve upon available apparatus can be made.

An oxygen-generating chemical additive, or air pump, or enriched oxygen gas can be incorporated.

Flavorings or colors can be added to enhance the flavor or appearance of the product. In particular, flavorings selected from the group not including sugars that are absorbed by the same GLUT transporters that transport DHAA are preferred, because sugars that are transported by the same transporters may competitively inhibit the transport of DHAA.

### EXAMPLES

### Example 1

Two cups of diced zucchini fruit were placed in a 2-quart blender with about ½ cup water and pureed for about 1 minute. Four commercially available vitamin C tablets containing 1000 mg AA each (Kirkland brand, Item 98268, Lot T00007 from Costco) were dissolved in ¼ cup hot water and added to the puree. Blender was capped and turned on to mix and the puree was tested periodically using a starch-iodine redox indicator for the presence of AA reducing activity. At about one minute, the redox indicator showed the presence of AA reducing activity. Within 10 minutes, the redox indicator showed that no more AA reducing activity was present in the puree, demonstrating that all of the AA in the puree had been oxidized to DHAA by oxygen that had been mixed into the puree from the air inside the blender, catalyzed by the AAO activity of the enzyme in the zucchini.

### Example 2

Four zucchini fruits about 6-8 inches long were purchased at a local grocery store. The shelf labeling indicated the fruits were a product of Mexico, as might be expected in Utah during the month of November when this experiment was conducted. Thus the fruit was not locally-grown and probably older (stored longer since picked) than known fresh-picked fruit. It is known that the AAO activity of zucchini fruit decreases during storage after being picked. The epicarp and outer portions of the mesocarp were peeled from the fruits, to the extent that approximately one-half of the weight of each fruit was included in the peelings. 300 grams of peelings were added to a blender with 100 grams purified water and pureed. 20 grams of the puree was removed and reserved for a “blank”. To the remaining puree (380 grams) was added 1.4 grams AA (as pure crystals approximately US mesh size 20-40) and the puree was mixed in the blender about 1 minute. The pH of the puree was measured and found to be 5.2, and a redox test indicated AA reducing activity in the puree. The puree was allowed to stand with periodic brief mixing for 35 minutes. At this point, the pH was 6.5 and the redox test showed that all AA was oxidized. A 20 gram portion was removed for a “test.” The blank and test solutions were centrifuged to remove the pulp so that pulp-free solutions could be spectrophotometrically analyzed. 25 uL of each of these solutions were diluted in 10 mL of 0.15 M phosphoric acid diluent, and the absorbance of each was determined using a UV spectrometer at 262 nm wavelength blanked against diluent. 2 mL of each dilution were combined with 2 mL of a TCEP reagent, incubated one hour, and then the absorbance at 262 nm of these solutions were determined as above (TCEP reagent reduces DHAA in the solution to AA; absorbance measurements at wavelengths where AA strongly absorbs, before and after treatment with TCEP, is a method known to practitioners in the art to quantify DHAA concentration by the differential absorption principle). The following absorption values were obtained (TCEP values are corrected for the X2 dilution):

| Sample | Abs 262 (mAU) | Abs post TCEP | Abs Difference |
|---|---|---|---|
| Blank | 0.060 | 0.068 | 0.008 |
| Test | 0.060 | 0.268 | 0.208 |

The Abs difference demonstrates that substantial amounts of DHAA are present in the “Test” puree as compared to the “Blank” puree.

### Example 3

One whole zucchini fruit weighing 235 grams was pureed in 120 mL water in a blender. The pH of the initial zucchini puree was 6.5. At time 0 minutes, 1.0 gram crystalline AA was added. After mixing 30 seconds, the pH was 5.0. At the times indicated in the table below, pH was recorded, iodine indicator redox result was recorded, and/or additional AA in the amounts indicated were added after testing the pH and redox status. The zucchini puree was continuously mixed in the blender during the entire time, and a cap in the top of the blender was left open to allow fresh air to be drawn into the vortex of the puree in the blender. The redox test results are reported + or −, ‘+’ indicating that AA reducing activity was detected, and ‘−’ indicating that AA reducing activity was not detected.

| Time (min.) | pH | Redox (+ or −) | AA added (g) |
|---|---|---|---|
| 0 | 6.5 | − | 1.0 |
| 5 | 5.0 | + | 0.0 |
| 10 | 5.0 | + | 0.0 |
| 15 | 5.3 | + | 0.0 |
| 20 | 6.6 | − | 0.5 |
| 25 | 6.4 | − | 0.5 |
| 29 | 6.3 | − | 0.5 |
| 33 | 5.9 | − | 0.5 |
| 36 | 5.9 | − | 0.5 |
| 40 | 5.6 | − | 0.5 |
| 45 | 5.4 | − | 0.5 |
| 48 | 5.2 | − | 0.5 |
| 52 | 5.0 | − | 0.5 |
| 56 | 5.0 | − | 0.5 |
| 59 | 4.8 | − | 0.5 |
| 62 | 4.7 | − | 0.5 |
| 67 | 4.5 | − | 0.5 |
| 71 | 4.4 | − | 0.5 |
| 77 | 4.3 | − | 0.5 |
| 89 | 4.5 | − | 0.0 |

Total AA added = 8.5 grams.
DHAA Max. Expected Concentration = 8.5 g/355 g solution = 2.4% w/w.

The product was split into three equal parts. Part 1 was kept in a closed container at room temperature. Part 2 was kept in a closed container at standard refrigerator temperature of 4 degrees C. Part 3 was kept in a closed container at common freezer temperature of minus 20 degrees C. Part 1 was tested immediately for DHAA concentration by the TCEP method previously discussed, and then after overnight storage (approx. 12 hours); Part 2 was tested after overnight storage (approx. 12 hours), and then after 13 days; Part 3 was tested after 13 days. Results are shown in the table below. Recovery column is the actual concentration divided by the maximum expected DHAA concentration of 2.4%, expressed as percent.

| | | DHAA | |
|---|---|---|---|
| Sample | Storage (hours) | Concentration | Recovery % |
| Part 1 (Immediate) | 0 | 2.27% | 95% |
| Part 1 (Room Temp) | 12 | 1.68% | 70% |
| Part 2 (Refrig.) | 12 | 2.14% | 89% |
| Part 2 (Refrig.) | 312 (13 d) | 1.27% | 53% |
| Part 3 (Freezer) | 312 (13 d) | 2.10% | 89% |

### CONCLUSION, RAMIFICATIONS AND SCOPE

While the above description contains much specificity, this should not be construed as limitations on the scope of the invention, but rather as exemplification of preferred embodiments. Accordingly, the scope of the invention should be determined not by the embodiments illustrated, but by the appended claims and their equivalents.

I claim:

1. A process comprising combining vegetable matter, or extracts thereof, which contain AAO enzyme, or synthetic AAO, with a form of reduced vitamin C, and oxygen, to create a mixture wherein said reduced vitamin C is oxidized to dehydroascorbic acid (DHAA).

2. A process comprising combining vegetable matter, or extracts thereof, which contain AAO enzyme, or synthetic AAO, with a form of reduced vitamin C, and oxygen, and copper ion in a concentration of about 0.01 mg/dl up to about 20 mg/dl, to create a mixture wherein said reduced vitamin C is oxidized to dehydroascorbic acid (DHAA).

3. A product manufactured according to the process of claim 2.

4. The process of claim 1 wherein a redox indicator is used to detect the disappearance of reducing activity in said mixture.

5. The process of claim 2 wherein a redox indicator is used to detect the disappearance of reducing activity in said mixture.

6. The process of claim 1 wherein said reduced vitamin C is added to said mixture in increments such that the pH of said mixture is maintained greater than about 4.0.

7. The process of claim 2 wherein said reduced vitamin C is added to said mixture in increments such that the pH of said mixture is maintained greater than about 4.0.

8. The process of claim 1 wherein said mixture is buffered such that the pH of said mixture is greater than about 4.0.

9. The process of claim 2 wherein said mixture is buffered such that the pH of said mixture is greater than about 4.0.

10. The process of claim 1 wherein said reduced vitamin C is in a concentration of about 0.1% w/w to about 20% w/w.

11. The process of claim 2 wherein said reduced vitamin C is in a concentration of about 0.1% w/w to about 20% w/w.

12. The process of claim 1 wherein the ratio of said vegetable matter to said reduced vitamin C is about 25:1 by weight.

13. The process of claim 2 wherein the ratio of said vegetable matter to said reduced vitamin C is about 25:1 by weight.
